(19) United States
(12) Patent Application Publication
(10) Pub. No.: US 2009/0304609 A1
(43) Pub. Date: Dec. 10, 2009

(54) FREEZE-DRIED MAKEUP PRODUCT, CORRESPONDING COMPOSITIONS AND METHOD FOR MAKING SAME

(75) Inventors: Jean-Luc Gabriel Allemand, Pelissanne (FR); Stephane Jacques Joseph Masson, Compiegne (FR); Anne Marie Catherine Simonnet (nee Carrere), Compiegne (FR); Sophie Caroline Vidal (nee Declomesnil), Aix en Provence (FR)

Correspondence Address: BRYAN CAVE POWELL GOLDSTEIN, ONE ATLANTIC CENTER FOURTEENTH FLOOR, 1201 WEST PEACHTREE STREET NW, ATLANTA, GA, 30309-3488, US

(73) Assignees: LYOFAL, Salon de Provence (FR); YVES SAINT LAURENT PARFUMS, Neuilly sur Seine (FR)

(21) Appl. No.: 11/910,368

(22) PCT Filed: Mar. 30, 2006
(86) PCT No.: PCT/FR2006/000694

§ 371 Date: Aug. 17, 2009

(30) Foreign Application Priority Data
Mar. 30, 2005 (FR) 0503107
Mar. 30, 2006 (FR) PCT/FR2006/000694

(51) Int. Cl. A61Q 17/04; A61Q 19/02; A61K 8/18; A61Q 1/06; A61Q 1/02

(52) U.S. Cl. 424/59; 424/62; 424/63; 424/64

(57) ABSTRACT

A base composition for makeup capable of being freeze-dried to form a makeup product, the base composition comprising an emulsion of an oily phase and an aqueous phase; fillers which are mainly insoluble; dispersing agents in sufficient amount to ensure dispersion of the fillers in the emulsion and formation, after being freeze-dried, of a network of cavities capable of trapping the fillings, the base composition comprising a proportion of fillers not less than 50 wt. %.

### PRIORITY CLAIM

This patent application is a U.S. National Phase of International Application No. PCT/FR2006/000694, filed Mar. 30, 2006, which claims priority to French Patent Application No. 0503107, filed Mar. 30, 2005, the disclosures of which are incorporated herein by reference in their entirety.

### FIELD

The present disclosure relates to makeup products, obtained from an emulsion of an oily phase and an aqueous phase comprising insoluble fillers in suspension intended, for example, to produce an optical, texturing or colouring effect.

The present disclosure relates, more particularly, to a base composition for makeup.

The present disclosure also relates to an intermediate composition for makeup, derived from the base composition.

The present disclosure also relates to a makeup product.

The present disclosure also relates to a method for manufacturing a makeup product.

The present disclosure also relates to dispersing agents within a makeup product.

### BACKGROUND

The use of emulsions is already known, for example, of an oily phase and an aqueous phase, for compounding cosmetic products intended to be applied to the skin. Thus, active principles, such as anti-oxidizing agents, vitamins or pigments, are incorporated in these emulsions. The emulsified form generally makes it possible to promote the homogeneous distribution of the active principles and the pigments.

Nevertheless, known emulsions present several disadvantages, derived, in particular, from their chemical instability. In particular, the emulsions are strongly oxidisable and often prove difficult to store.

In order to overcome these disadvantages, it is known, in particular, from International Patent Publication No. WO 2001/082886, to compound lyophilised cosmetic compositions, more stable than traditional emulsions, and intended to be applied to the skin after re-hydration.

It is also known to use compacted makeup powders, formed from a mixture of powders with an oily binder and formed, for example, by compression. Known compacted powders generally have storage properties superior to those of the emulsions, but also suffer from several disadvantages.

In particular, it is not possible, with traditional compacted powders, to manufacture products presenting a high percentage of insoluble fillers, and in particular pigments or mothers of pearl.

Thus, when the percentage of pigments or mothers of pearl increases within the product, the manufacture and compaction of the product become complicated, indeed impossible, to carry out on an industrial scale, taking account of the requirements for quality and productivity. In addition, the known compacted powders are generally brittle and often present poor resistance to shocks, in particular, when the level of fillers incorporated is greater than 40%.

### SUMMARY

The present disclosure describes several exemplary embodiments of the present invention.

One aspect of the present disclosure provides a base composition for makeup capable of being lyophilised to form a makeup product, the base composition comprising: a) an emulsion of an oily phase and an aqueous phase; b) fillers which are predominantly insoluble; and c) dispersing agents, in sufficient quantity to ensure the dispersion of the fillers in the emulsion and to form, after lyophilisation, a network of cavities capable of trapping the fillers; the base composition including a proportion of fillers greater than or equal to 50%. Another aspect of the present disclosure provides an intermediate composition for makeup capable of being lyophilised to form a makeup product and including a mixture comprising: a) a base composition comprising i) an emulsion of an oily phase and an aqueous phase, ii) fillers which are predominantly insoluble, and iii) dispersing agents, in sufficient quantity to ensure the dispersion of the fillers in the emulsion and to form, after lyophilisation, a network of cavities capable of trapping the fillers, the base composition including a proportion of fillers greater than or equal to 50%; and b) a quantity of water sufficient so that the intermediate composition has a proportion of water greater than 50% by weight.

A further aspect of the present disclosure provides a lyophilised makeup product obtained by lyophilisation of a base composition comprising: a) an emulsion of a oily phase and an aqueous phase; b) fillers which are predominantly insoluble; and c) dispersing agents, in sufficient quantity to ensure the dispersion of the fillers in the emulsion and to form, after lyophilisation, a network of cavities capable of trapping the fillers; the base composition comprising a proportion of fillers greater than or equal to 50%.

An additional aspect of the present disclosure provides a process for manufacturing a lyophilised makeup product, comprising: a) preparing either a base composition or an intermediate composition obtained by dilution of the base composition, the composition comprising an emulsion in aqueous phase and a oily phase as well as fillers which are predominantly insoluble in suspension in the emulsion, wherein the base composition has a proportion of fillers greater than or equal to 50% by weight; and b) lyophilizing either the base composition or the intermediate composition; wherein step a) includes a sub-step of incorporating dispersing agents in the emulsion in sufficient quantity to form, during step b), a network capable of trapping the fillers.

Yet another aspect of the present disclosure provides a lyophilised makeup product obtained by lyophilisation of an intermediate composition comprising: a) a base composition comprising: i) an emulsion of a oily phase and an aqueous phase; ii) fillers which are predominantly insoluble; and iii) dispersing agents, in sufficient quantity to ensure the dispersion of the fillers in the emulsion and to form, after lyophilisation, a network of cavities capable of trapping the fillers; the composition including a proportion of fillers greater than or equal to 50%; and b) a quantity of water sufficient so that the intermediate composition has a proportion of water greater than 50% by weight.

The features of the present disclosure remedy the various disadvantages enumerated previously and propose a composition, in particular, for the base, comprising predominantly insoluble fillers, which will make it possible to obtain a makeup product which, while presenting a high making-up power, is stable over time, unitary and resistant on the mechanical plane.

Another feature of the present disclosure is a base composition making it possible to obtain a makeup product comprising a high proportion of insoluble fillers while presenting good dimensional and structural stability as well as good resistance to shocks.

Another feature of the present disclosure is a base composition making it possible to obtain a makeup product that is not oily to the touch.

Another feature of the present disclosure is a base composition making it possible to obtain a makeup product that can be used dry or after re-hydration.

Another feature of the present disclosure is a base composition making it possible to obtain a makeup product presenting a very good hold on the skin while being easy to remove, without necessitating a special solvent other than water.

The features of the present disclosure propose an intermediate composition, derived from the base composition, intended to facilitate the manufacture and forming, by lyophilisation, of a makeup product.

The features of the present disclosure propose a lyophilised makeup product which, while presenting a high making-up power, is particularly stable over time, unitary and non-brittle.

Another feature of the present disclosure proposes a makeup product comprising a high proportion of incorporated insoluble fillers while presenting good mechanical resistance, in particular, to shocks.

Another feature of the present disclosure proposes a makeup product the density of which will be particularly low.

The features of the present disclosure propose a manufacturing method for a makeup product with a base of predominantly insoluble fillers making it possible to obtain a product which, while presenting a high making-up power, is stable over time, unitary, non-brittle and presents good properties of mechanical resistance.

### DETAILED DESCRIPTION

Other advantages and features of the present disclosure will appear in more detail upon reading of the description that follows.

The base composition of the present disclosure is intended to be lyophilised to form a lyophilised makeup product, such as eye shadow, blush, lipstick or foundation, intended, for example, for makeup of the face and/or body.

According to the present disclosure, the base composition comprises an emulsion of two immiscible phases, and, more precisely, an emulsion of an oily phase and an aqueous phase.

The base composition also comprises predominantly insoluble fillers.

For purposes of the present disclosure, the term “predominantly insoluble fillers” is defined as fillers, i.e., particles that are colourless or coloured, inorganic or organic, natural or synthetic forming a pulverulent phase, and which are either entirely insoluble or partially soluble in the emulsion, the soluble part of the fillers being in the minority with respect to the insoluble part, which is in the majority. Particles such as lacquers, for example, may be involved, formed from a supporting means on which a dye is deposited, the dye being capable of dissolving at least partially in one of the phases of the emulsion, namely, the oily phase and/or the aqueous phase, and the supporting means being insoluble.

The predominantly insoluble fillers are advantageously chosen from one or more of the following groups consisting of:

inorganic or organic pigments, natural or synthetic,

lacquers;

nacreous particles;

fillers with a texturing effect; and/or

fillers with an optical effect.

The inorganic or organic synthetic pigments or the natural pigments advantageously constitute a coloured pulverulent phase and can be chosen from the following list of compounds consisting of, but not limited to, chromium oxides, ferric ferrocyanides, guaiazulene, iron oxides, ultramarines, azurite, malachite, lazurite, manganese violets, titanium oxides, zinc oxides, plant black, azo group pigments (D&C red 34, etc.), graphite, and/or hydrophilic or hydrophobic coloured fibres, of synthetic or natural origin, inorganic or organic. These fibres may also be long, short, unitary or organised, and their cross-section may be circular or polygonal.

Such pigments make it possible to obtain a decorative makeup for the face and/or body.

The lacquers, used as fillers, are advantageously pigmentary colouring substances obtained from water-soluble or lipid-soluble dyes by adsorption on an inert inorganic supporting medium, such as alumina hydrate, or on a mordant, i.e., a substance (such as a fibre) used to fix the dye.

The water-soluble dyes mentioned above can be formed from natural or synthetic dyes chosen preferably from the following compounds: annatto, hypoxanthine, caramel, carotenoids, pastel blue, carmine, garance red, garance orange, Brazilwood red, logwood violet, goldenrod yellow, dyer's weed yellow, cosmos orange, buckthorn orange, capsanthin, capsorubin, henna, chlorophyll green, tannin black, extract of crocus sativus flower, extract of coptis chinensis rhizome, extract of vitis vinifera juice, curry plant seed, gardenia florida extract, tannin beige, tropical indigo blue, nitro group dyes (ex: D&C Yellow 7), azo group dyes, xanthene group dyes, quinoline group dyes, anthroquinone group dyes, indigoid group dyes, phthalocyanine group dyes, and the like.

Advantageously, the nacreous particles (also called mothers of pearl) are formed from insoluble particles, coloured or not, intended to provide highlights and iridescence. The nacreous particles are thus preferentially chosen from the following particles: bismuth oxychloride, metallic particles (bronze powder, aluminium powder, copper powder), guanine, silver, gold, natural or synthetic mica (fluorphlogopite) with single or multiple coatings (titanium dioxide, metallic oxides, organic pigments, dyes), borosilicates with single or multiple coatings, silica with single or multiple coatings, metallic particles with single or multiple coatings, iron oxides with single or multiple coatings, silicas with single or multiple coatings, decorative coloured glitter specks made of polyester or aluminium epoxy of various shapes, liquid crystals, and the like.

The fillers with a texturing effect are preferably uncoloured and used as texture agents. These fillers may be either of natural (plant or inorganic) or synthetic origin. As insoluble fillers with texturing effect, the following substances may thus be used: talc, mica, biotite, acrylate copolymers, clay, kaolin, boron nitride, polymethylmethacrylate, polyurethane, bentonite, modified or not, chalk, amethyst powder, alumina, barium sulphate, silica, modified or not, diamond powder, glass beads, hectorite, hydroxyapatite, calcite, dolomite, diatoms, hydrophobic silica, loess, fluorphlogopite, magnesium aluminium hydroxide carbonate, magnesium aluminium zinc hydroxide carbonate, magnesium nitrate, magnesium oxide, magnesium and potassium fluorosilicate, magnesium and sodium fluorosilicate, magnesium potassium silicon fluoride hydroxide oxide, montmorillonite, Moroccan lava clay, mother of pearl, silk powder, opal powder, perfluoroalkylsilyl mica, quartz, phonolite, hectorite, modified or not, rhodochrosite, ruby powder, sand, sapphire powder, schist extract, zeolite, smithsonite, topaz powder, tourmaline powder, zirconium powder, volcanic powders (cinders, stone, sand), borosilicate, alumina hydrate, calcium carbonate, calcium sulphate, magnesium carbonate, silicone powders, cellulose powder, starch powder, modified or not, nylon 6, nylon 6/12, nylon 11, nylon 12, nylon 66, polymethylsilsesquioxane, amylodextrin, oat flour, soy flour, bamboo powders, mother of pearl powder, rice powder, PTFE, polyethylene, rayon, wheat flour, wood powders, corn flour, fruit zest powders, dextrins, cyclodextrins, maltodextrins, hydrophilic or hydrophobic uncoloured fibres, of synthetic or natural origin, inorganic or organic, these fibres capable of being long, short, unitary or organised and their cross-section can be circular or polygonal, and/or dendritic polymers, HDI/trimethylol hexylactone crosspolymer, and/or calcium sodium phosphosilicate and mica (Bioglass), and the like.

The fillers with optical effect are preferentially chosen for their optical properties and are preferentially chosen from the list of the following substances: pigments with optical effects (light-diffusing soft focus pigments), photosensitive pigments, thermochromic pigments, photochromic pigments, optical brightening agents pH sensitive pigments, and the like.

Preferentially, the oily phase constitutes the dispersed phase of the emulsion, and is presented in the form of drops, whereas the aqueous phase constitutes the continuous phase. It may thus be a matter of an emulsion of an oil in water. It is quite obviously conceivable, without leaving the framework of the present disclosure, that the emulsion may be formed by an emulsion of water in an oil, the water then forming the dispersed phase and the oil forming the continuous phase of the emulsion.

Moreover, without leaving the framework of the present disclosure, it is also conceivable to construct an emulsion of silicone (oily phase) in water or of water in silicone, the water and silicone being capable of forming, respectively, either the dispersed phase or the continuous phase.

According to one exemplary embodiment of the present disclosure, the base composition comprises dispersing agents in a quantity sufficient to ensure, on the one hand, the dispersion of the predominantly insoluble fillers in the emulsion and, on the other hand, to form, after lyophilisation, a network of cavities suitable for trapping and imprisoning the fillers.

The dispersing agents are advantageously chosen for their capacity to interact with the fillers, on the one hand, to avoid their agglomeration and thus obtain a base composition and a final product the most homogeneous possible, and, on the other hand, for their capacity to form, in lyophilised form, a network capable of trapping the fillers within cavities.

Preferentially, the dispersing agents are formed from hydrocolloids, i.e., colloids soluble in water, chosen for their high dispersing power, in particular, with respect to the fillers.

The hydrocolloids are preferably chosen from the group consisting of polyacrylamide isoparaffin laureth-7 (SEPIGEL™), the silicate of aluminium and magnesium (VEEGUM™), and xanthan gum.

Even more preferentially, the hydrocolloids are formed predominantly by the silicate of magnesium and aluminium.

In order to obtain a lyophilised product presenting a network of cavities sufficient in number to trap the fillers, the weight ratio of concentration between the dispersing agents and the fillers ([dispersing agents]/[fillers]) is preferentially between 0.01 and 0.5 and even more preferentially between 0.01 and 0.1.

The fillers thus imprisoned within the cavities are progressively released as the hydration of the product, and, in particular, the superficial layers of the product, proceeds. Thus, the product constitutes a porous structure capable, by means of the network of cavities, of being easily re-hydrated. The fillers can also be progressively released during direct application of the product to the skin. The lyophilised cosmetic product thus obtained is particularly stable and does not present a fragile and brittle character, even for high proportions of fillers.

Advantageously, the cavities formed serve also to trap the oily phase; and the weight ratio of concentration between the dispersing agents and the oily phase ([dispersing agents]/[oily phase]) is advantageously between 0.01 and 1, and preferably between 0.2 and 0.5, and even more preferentially between 0.2 and 0.4. The imprisonment of the oily phase within the cavities thus makes it possible to obtain a lyophilised makeup product in the form of a dry emulsion, non-oily to the touch and very pleasant to use.

Advantageously, the weight ratio of concentration between the oily phase and the fillers ([oily phase]/[fillers]) is between 0.1 and 3.5, preferably between 0.1 and 1 and even more preferentially between 0.1 and 0.5. Thus, contrary to traditional formulations (emulsions or compacted powders), the lyophilised makeup product advantageously presents a proportion of predominantly insoluble fillers greater than the proportion of oily phase, while preserving a stable and non-brittle character.

For the emulsions of an oily phase in an aqueous phase, for which the oily phase constitutes the dispersed phase and the aqueous phase constitutes the continuous phase, the proportion of oily phase in the base composition is preferentially less than 20% by weight, still more preferentially less than 10% by weight, in particular, for the eye shadow and blush applications. The lyophilised makeup product advantageously comprises a proportion of oily phase less than 40% by weight, preferentially less than 30% by weight and even more preferentially less than 15% by weight. The low percentage of oily phase makes possible rapid removal of the product, for example, by cleaning the skin with a simple cotton ball soaked with water.

According to another exemplary embodiment of the present disclosure, the base composition comprises a proportion of fillers appreciably greater than or equal to 50% by weight, and even more preferentially appreciably greater than or equal to 60% by weight, a proportion of fillers appreciably greater than or equal to 65% by weight also giving excellent results. This large proportion of fillers in the base composition makes it possible to obtain a lyophilised makeup product presenting a very large proportion of fillers by comparison to traditional formulations such as emulsions or compacted powders, and therefore a high making-up power.

According to another exemplary embodiment of the present disclosure, the base composition comprises a proportion of water less than 50% by weight and preferably less than 40% by weight. The base composition thus takes on the appearance of a thick paste, compact and non-fluid, unstructured and not smooth, presenting in the form of aggregates.

According to still another exemplary embodiment of the present disclosure, the base composition also comprises at least one active principle chosen from one or more of the following groups consisting of:

self-tanning agent;

anti-UVA and anti-UVB sun screens and filters;

anti-free radical agent;

bleaching agent;

hydrating agent; and/or

treating agent.

The auto-tanning agents may include dihydroxyacetone and/or erythrulose.

The sun screens and filters are preferentially chosen from the following substances: screens (micronized titanium dioxides, micronized zinc oxides), chemical filters (para-aminobenzoic acid (PABA)), cinnamic esters, salicylates, benzimidazole derivatives, etc.) and/or filtering molecules of natural origin. The anti-free radical agents are preferentially chosen from the list of the following compounds: flavonoids, polyphenols, procyanidolic oligomers, lycopene, carotenoids, vitamin E, and/or vitamin C. The bleaching agents are preferentially formed from derivatives of vitamin C or plant extracts.

The hydrating agents are preferentially chosen from the following compounds: humectants (glycerol, sorbitol, lactic acid, lactates, alpha-hydroxyacids, etc.), components of the NMF (Natural Moisturising Factor) (urea, sodium pyrrolidone carboxylate, amino acids, sugars), hydrophilic film-forming agents (hyaluronic acid, glycosaminoglycans, substantive polymers), and/or hydrophobic film-forming agents (duplicate of the epidermal lipids).

The treating agents are preferentially chosen from the list of the following substances: trace elements, lipid-soluble and water-soluble vitamins, plant extracts, minerals, extracts obtained by biotechnology, synthetic actives, macromolecules, nonsaponifiable matter, essential oily acids, micro-circulation activators, agents regulating sebaceous function, soothers. The active principles thus make it possible to obtain a makeup product that is not only decorative but also treating, solar and/or concealing for the face and/or body.

The base composition and the lyophilised makeup product of the present disclosure can also comprise any type of adjuvant known, used for cosmetic or makeup applications. Several examples of formulations of the base composition will now be described for applications as eye shadow, blush, foundation and lipstick.

### EXAMPLES

### Example 1

### Lyophilised Base Composition for Eye Shadow or Blush

A base composition for makeup that is suitable for being lyophilised to form a lyophilised makeup product intended for application to the skin as eye shadow or blush is realised.

A first variant of realisation of this base composition is described in Table 1 below, wherein:

components No. 7 to 10 form the oily phase of the emulsion;

components No. 11, 12, 14 and 16 to 19 form the predominantly insoluble fillers in the emulsion; and

components No. 6, 13 and 15 form dispersing agents and more precisely hydrocolloids.

The term “INCI,” mentioned in Table 1, refers to International Nomenclature of Cosmetic Ingredients.

TABLE 1

| | | % by |
|---|---|---|
| No. | INCI NAMES | Weight |
| 1 | WATER | 35.60 |
| 2 | BUTYLENE GLYCOL (solvent) | 1.00 |
| 3 | METHYLPARABEN (preservative) | 0.19 |
| 4 | PROPYLPARABEN (preservative) | 0.19 |
| 5 | BUTYLPARABEN (preservative) | 0.19 |
| 6 | XANTHAN GUM (hydrocolloid) | 1.00 |
| 7 | SORBITAN STEARATE (oily phase) | 1.25 |
| 8 | CAPRYLIC/CAPRIC TRIGLYCERIDE (oily phase) | 2.00 |
| 9 | DIPENTAERYTHRITYL HEXAHYDROXYSTEARATE/ | 1.65 |
| | HEXASTEARATE/HEXAROSINATE (oily phase) | |
| 10 | OCTYLDODECYL STEAROYL STEARATE (oily phase) | 3.20 |
| 11 | ZEA MAYS STARCH (filler) | 2.00 |
| 12 | ALUMINIUM STARCH OCTENYLSUCCINATE (filler) | 2.00 |
| 13 | POLYACRYLAMIDE AND ISOPARAFFIN AND LAURETH-7 | 2.00 |
| | (hydrocolloid) | |
| 14 | TALC (filler) | 0.80 |
| 15 | MAGNESIUM ALUMINIUM SILICATE (hydrocolloid) | 5.00 |
| 16 | MICA (filler) | 0.50 |
| 17 | NYLON-12 (filler) | 1.00 |
| 18 | SILICA as silica beads (filler) | 0.20 |
| 19 | MICA/IRON OXIDE/TITANIUM DIOXIDE (filler | 40.23 |
| | (mother of pearl)) | |

Particularly advantageously, the weight ratio of concentration between the hydrocolloids and the fillers ([hydrocolloids]/[fillers]) is equal to 0.17 for this formulation.

The weight ratio of concentration between the hydrocolloids and the oily phase ([hydrocolloids]/[oily phase]) is equal to 0.99, and the weight ratio of concentration between the oily phase and the fillers ([oily phase]/[fillers]) is equal to 0.17.

A second preferential variant of realisation of this base composition for eye shadow or blush is described in Table 2 below, in which the same ingredients are found as in Table 1, but in different proportions.

TABLE 2

| | | % by |
|---|---|---|
| No. | INCI NAMES | Weight |
| 1 | WATER | 35.60 |
| 2 | BUTYLENE GLYCOL (solvent) | 1.00 |
| 3 | METHYLPARABEN (preservative) | 0.19 |
| 4 | PROPYLPARABEN (preservative) | 0.19 |
| 5 | BUTYLPARABEN (preservative) | 0.19 |
| 6 | XANTHAN GUM (hydrocolloid) | 0.03 |
| 7 | SORBITAN STEARATE (oily phase) | 1.25 |
| 8 | CAPRYLIC/CAPRIC TRIGLYCERIDE (oily phase) | 2.00 |
| 9 | DIPENTAERYTHRITYL HEXAHYDROXYSTEARATE/ | 1.65 |
| | HEXASTEARATE/HEXAROSINATE (oily phase) | |
| 10 | OCTYLDODECYL STEAROYL STEARATE (oily phase) | 3.20 |
| 11 | ZEA MAYS STARCH (filler) | 2.00 |
| 12 | ALUMINIUM STARCH OCTENYLSUCCINATE (filler) | 2.00 |
| 13 | POLYACRYLAMIDE AND ISOPARAFFIN AND LAURETH-7 | 0.80 |
| | (hydrocolloid) | |
| 14 | TALC (filler) | 2.70 |
| 15 | MAGNESIUM ALUMINIUM SILICATE (hydrocolloid) | 2.00 |
| 16 | MICA (filler) | 2.50 |
| 17 | NYLON-12 (filler) | 1.00 |
| 18 | SILICA as silica beads (filler) | 0.20 |
| 19 | MICA/IRON OXIDE/TITANIUM DIOXIDE (filler | 41.50 |
| | (mother of pearl)) | |

In particular, the weight ratio of concentration between the hydrocolloids and the fillers ([hydrocolloids]/[fillers]) is equal to 0.05 for this formulation.

The weight ratio of concentration between the hydrocolloids and the oily phase ([hydrocolloids]/[oily phase]) is equal to 0.35, and the weight ratio of concentration between the oily phase and the fillers ([oily phase]/[fillers]) is equal to 0.15.

This base composition thus makes it possible to obtain, after removal of the water by lyophilisation, a lyophilised makeup product (eye shadow or blush) comprising a percentage of mothers of pearl greater than 65% and a percentage of fillers (including the mothers of pearl and the fillers with a texturing effect, such as talc or mica) greater than 70% and up to 80% for the second variant of realisation. The percentage of oily phase in the finished product is, in addition, preferentially less than 20%, and even more preferentially less than 15%.

For the makeup products, such as blush or eye shadow, the weight ratio between the hydrocolloids (dispersing agents) and the fillers ([hydrocolloids]/[fillers]) will preferably be between 0.01 and 0.2 and even more preferentially between 0.01 and 0.1, while the weight ratio of concentration between the hydrocolloids and the oily phase ([hydrocolloids]/[oily phase]) will preferentially be between 0.2 and 1 and even more preferentially between 0.2 and 0.5, the weight ratio of concentration between the oily phase and the fillers ([oily phase]/[fillers]) being preferentially between 0.1 and 0.2.

The compositions for eye shadow or blush may also comprise active principles such as those set forth previously, without leaving the framework of the present disclosure.

### Example 2

### Base Composition for Foundation Cream

A base composition designed to be lyophilised is made in order to obtain a lyophilised makeup product for use as foundation cream.

One first variant of this base composition for lyophilised foundation cream is described in Table 3 below, in which are to be found substantially the same constituents as in Tables 1 or 2 but in different proportions.

In particular, the proportion of the constituent 18, formed by the mother of pearl particles, is lower in the foundation cream than in the eye shadow; and the proportion of the oily phase is, on the contrary, substantially higher in the foundation cream with respect to the eye shadow.

TABLE 3

| | | % IN |
|---|---|---|
| N° | INCI NAMES | WEIGHT |
| 1 | WATER | 51.73 |
| 2 | BUTYLENE GLYCOL (solvent) | 0.72 |
| 3 | METHYLPARABEN (preservative) | 0.15 |
| 4 | PROPYLPARABEN (preservative) | 0.15 |
| 5 | BUTYLPARABEN (preservative) | 0.15 |
| 6 | XANTHAN GUM (hydrocolloid) | 1.00 |
| 7 | SORBITAN STEARATE (oily phase) | 5.00 |
| 8 | CAPRYLIC/CAPRIC TRIGLYCERIDE (oily phase) | 10.00 |
| 9 | DIPENTAERYTHRITYL HEXAHYDROXYSTEARATE/ | 5.00 |
| | HEXASTEARATE/HEXAROSINATE | |
| 10 | OCTYLDODECYL STEAROYL STEARATE | 10.00 |
| 11 | ZEA MAYS STARCH (filler) | 1.00 |
| 12 | ALUMINIUM STARCH OCTENYLSUCCINATE (filler) | 1.00 |
| 13 | POLYACRYLAMIDE AND ISOPARAFFIN AND LAURETH-7 | 4.00 |
| | (hydrocolloid) | |
| 14 | TALC (filler) | 1.00 |
| 15 | MAGNESIUM ALUMINIUM SILICATE (hydrocolloid) | 0.10 |
| 16 | MICA (filler) | 0.50 |
| 17 | NYLON-12 (filler) | 0.50 |
| 18 | SILICE as silica beads (filler) | 2.00 |
| 19 | MICA/IRON OXIDE/TITANIUM OXIDE (fillers) | 6.00 |
| | (mother of pearl) | |

For this first variant, the proportion by weight of the concentration between the hydrocolloids and the oily phase ([hydrocolloids]/[oily phase]) is around 0.17.

The proportion by weight of the concentration between the hydrocolloids and the fillers ([hydrocolloids]/[fillers]) is around 0.41 and the proportion by weight of the concentration between the oily phase and the fillers ([oily phase]/[fillers]) is around 2.41.

A second variant of the preferred exemplary embodiment of this base composition for foundation cream is described in Table 4 below, where the same ingredients may be found as in Table 3, but in different proportions.

TABLE 4

| | | % IN |
|---|---|---|
| N° | INCI NAMES | WEIGHT |
| 1 | WATER | 49.00 |
| 2 | BUTYLENE GLYCOL (solvent) | 0.72 |
| 3 | METHYLPARABEN (preservative) | 0.15 |
| 4 | PROPYLPARABEN (preservative) | 0.15 |
| 5 | BUTYLPARABEN (preservative) | 0.15 |
| 6 | XANTHAN GUM (hydrocolloid) | 0.03 |
| 7 | SORBITAN STEARATE (oily phase) | 2.00 |
| 8 | CAPRYLIC/CAPRIC TRIGLYCERIDE (oily phase) | 3.50 |
| 9 | DIPENTAERYTHRITYL HEXAHYDROXYSTEARATE/ | 3.50 |
| | HEXASTEARATE/HEXAROSINATE | |
| 10 | OCTYLDODECYL STEAROYL STEARATE | 5.00 |
| 11 | ZEA MAYS STARCH (filler) | 1.20 |
| 12 | ALUMINIUM STARCH OCTENYLSUCCINATE (filler) | 1.20 |
| 13 | POLYACRYLAMIDE AND ISOPARAFFIN AND LAURETH-7 | 1.50 |
| | (hydrocolloid) | |
| 14 | TALC (filler) | 2.20 |
| 15 | MAGNESIUM ALUMINIUM SILICATE (hydrocolloid) | 2.80 |
| 16 | MICA (filler) | 3.50 |
| 17 | NYLON-12 (filler) | 1.40 |
| 18 | SILICE as silica beads (filler) | 2.00 |
| 19 | MICA/IRON OXIDE/TITANIUM OXIDE (fillers) | 20.00 |
| | (mother of pearl) | |

In particular, the proportion by weight between the hydrocolloids and the fillers ([hydrocolloids]/[fillers]) is equal to 0.13 for this formulation.

The proportion by weight between the hydrocolloids and the oily phase ([hydrocolloids]/[oily phase]) is equal to 0.31, and the proportion by weight between the hydrocolloids and the fillers ([hydrocolloids]/[fillers]) is equal to 0.44.

This base composition for foundation cream thus permits, after elimination of the water by lyophilisation, a lyophilised makeup product (foundation cream) to be obtained comprising a percentage of fillers (including mother of pearl and texturing effect fillers such as talc or mica) that is greater than 20% and preferably greater than 60% and up to 62% for the second variant of the exemplary embodiment. The percentage of oily phase in the finished product is higher than in Example 1, but preferably remains lower than 30%.

For foundation cream type makeup products, the proportion by weight between the hydrocolloids (dispersing agents) and the fillers ([hydrocolloids]/[fillers]) will preferably be between 0.01 and 0.5, and even more preferably between 0.01 and 0.2, whereas the proportion by weight of the concentration between the hydrocolloids and the oily phase ([hydrocolloids]/[oily phase]) will preferably be between 0.2 and 1, and even more preferably between 0.2 and 0.5, wherein the proportion by weight of the concentration between the oily phase and the fillers ([oily phase]/[fillers]) will preferably be between 0.1 and 3.5 and even more preferably between 0.1 and 1.

The compositions for foundation cream may also comprise active principles such as those mentioned previously, without leaving the scope of the present disclosure.

### Example 3

### Base Composition for Lipstick

A base composition designed to be lyophilised is made in order to obtain a lyophilised makeup product for use as lipstick.

One first variant of this base composition is described in Table 5 below, in which are to be found substantially the same constituents as in Examples 1 or 2 but in slightly different proportions.

In particular, the proportion of the oily phase in the base composition of the lipstick is slightly higher than in the eye shadow described in Example 1.

TABLE 5

| | | % IN |
|---|---|---|
| N° | INCI NAMES | WEIGHT |
| 1 | WATER | 45.33 |
| 2 | BUTYLENE GLYCOL (solvent) | 0.60 |
| 3 | METHYLPARABEN (preservative) | 0.19 |
| 4 | PROPYLPARABEN (preservative) | 0.19 |
| 5 | BUTYLPARABEN (preservative) | 0.19 |
| 6 | XANTHAN GUM (hydrocolloid) | 1.00 |
| 7 | SORBITAN STEARATE (oily phase) | 2.00 |
| 8 | CAPRYLIC/CAPRIC TRIGLYCERIDE (oily phase) | 5.00 |
| 9 | DIPENTAERYTHRITYL HEXAHYDROXYSTEARATE/ | 5.00 |
| | HEXASTEARATE/HEXAROSINATE (oily phase) | |
| 10 | OCTYLDODECYL STEAROYL STEARATE (oily phase) | 10.00 |
| 11 | ZEA MAYS STARCH (filler) | 1.20 |
| 12 | ALUMONIUM STARCH OCTENYLSUCCINATE (filler) | 1.20 |
| 13 | POLYACRYLAMIDE AND ISOPARAFFIN AND LAURETH-7 | 4.00 |
| | (hydrocolloid) | |
| 14 | TALC (filler) | 1.20 |
| 15 | MAGNESIUM ALUMINIUM SILICATE (hydrocolloid) | 5.00 |
| 16 | MICA (filler) | 3.50 |
| 17 | NYLON-12 (filler) | 1.40 |
| 18 | SILICE silica beads type (filler) | 3.00 |
| 19 | MICA/IRON OXIDE/TITANIUM OXIDE (fillers) | 10.00 |
| | (mother of pearl) | |

For this first variant, the proportion by weight of the concentration between the hydrocolloids and the oily phase ([hydrocolloids]/[oily phase]) is around 0.45.

The proportion by weight of the concentration between the hydrocolloids and the fillers ([hydrocolloids]/[fillers]) is around 0.45, and the proportion by weight of the concentration between the oily phase and the fillers ([oily phase]/[fillers]) is around 1.

A second variant of the preferred exemplary embodiment of this base composition for lipstick is described in Table 6 below, where the same ingredients may be found as in Table 5, but in different proportions.

TABLE 6

| | | % IN |
|---|---|---|
| N° | INCI NAMES | WEIGHT |
| 1 | WATER | 29.00 |
| 2 | BUTYLENE GLYCOL (solvent) | 0.60 |
| 3 | METHYLPARABEN (preservative) | 0.19 |
| 4 | PROPYLPARABEN (preservative) | 0.19 |
| 5 | BUTYLPARABEN (preservative) | 0.19 |
| 6 | XANTHAN GUM (hydrocolloid) | 0.03 |
| 7 | SORBITAN STEARATE (oily phase) | 2.00 |
| 8 | CAPRYLIC/CAPRIC TRIGLYCERIDE (oily phase) | 3.50 |
| 9 | DIPENTAERYTHRITYL HEXAHYDROXYSTEARATE/ | 3.50 |
| | HEXASTEARATE/HEXAROSINATE (oily phase) | |
| 10 | OCTYLDODECYL STEAROYL STEARATE (oily phase) | 5.00 |
| 11 | ZEA MAYS STARCH (filler) | 1.20 |
| 12 | ALUMONIUM STARCH OCTENYLSUCCINATE (filler) | 1.20 |
| 13 | POLYACRYLAMIDE AND ISOPARAFFIN AND LAURETH-7 | 1.50 |
| | (hydrocolloid) | |
| 14 | TALC (filler) | 1.20 |
| 15 | MAGNESIUM ALUMINIUM SILICATE (hydrocolloid) | 2.80 |
| 16 | MICA (filler) | 3.50 |
| 17 | NYLON-12 (filler) | 1.40 |
| 18 | SILICE as silica beads (filler) | 3.00 |
| 19 | MICA/IRON OXIDE/TITANIUM OXIDE (fillers) | 40.00 |
| | (mother of pearl) | |

In particular, the proportion by weight between the hydrocolloids and the fillers ([hydrocolloids]/[fillers]) is equal to 0.08 for this formulation.

The proportion by weight between the hydrocolloids and the oily phase ([hydrocolloids]/[oily phase]) is equal to 0.31, and the proportion by weight between the hydrocolloids and the fillers ([hydrocolloids]/[fillers]) is equal to 0.27.

This base composition for lipstick thus permits, after elimination of the water by lyophilisation, a lyophilised makeup product (lipstick) to be obtained comprising a percentage of fillers (including mother of pearl and texturing effect fillers, such as talc or mica) that is greater than 40%, and preferably greater than 60% and up to 72% for the second variant of the exemplary embodiment. The percentage of oily phase in the finished product is higher than in Example 1, but preferably remains lower than 40% and even more preferably lower than 25-30%.

For lipstick type makeup products, the proportion by weight between the hydrocolloids (dispersing agents) and the fillers ([hydrocolloids]/[fillers]) will preferably be between 0.01 and 0.5, and even more preferably between 0.01 and 0.2, whereas the proportion by weight of the concentration between the hydrocolloids and the oily phase ([hydrocolloids]/[oily phase]) will preferably be between 0.2 and 1, and even more preferably between 0.2 and 0.5, wherein the proportion by weight of the concentration between the oily phase and the fillers ([oily phase]/[fillers]) will preferably be between 0.1 and 3.5 and even more preferably between 0.1 and 1.

The compositions for lipstick may also comprise active principles such as those mentioned previously, without leaving the scope of the present disclosure.

The present disclosure also relates to an intermediate composition derived from the base composition and capable of being lyophilised to form a makeup product and which comprises a mixture comprising the base composition previously described and an adequate quantity of water so that the proportion of water in the intermediate composition is greater than 50% in weight and preferably greater than 60% in weight.

The dilution of the base composition to obtain the intermediate composition thus permits a fluid formula to be obtained that is capable of being easier to spread onto the supports, for example, in trays, than the paste forming the base composition.

Preferably, the base composition is diluted to 50% to obtain the intermediate composition. Advantageously, the intermediate composition has an apparent viscosity of between 100 and 150 mPa·s at 25° C., for a shear gradient of 125 s⁻¹. The apparent viscosity is measured by a HAAKE VT 500 type viscometer (mobile NV, speed 4). The intermediate composition advantageously forms a non Newtonian fluid.

The intermediate composition obtained by dilution of the base composition is furthermore thermodynamically unstable. Consequently, when the intermediate composition is centrifuged at ambient temperature, at a speed of 6,000 rpm for 5 minutes, a mixture is obtained that is formed by two distinct and separate phases, which illustrates the unstable character of the intermediate composition. On the contrary, the centrifugation of a classic emulsion for makeup does not modify the appearance of the emulsion, which conserves its homogeneity, from whence its stability is deduced.

Examples of intermediate compositions of the present disclosure may be obtained by dilution, for example at 50%, of the base compositions illustrated in Examples 1-3.

The present disclosure also relates to a lyophilised makeup product obtained by lyophilisation of the base composition previously described. The lyophilised makeup product thus obtained advantageously forms a dry emulsion, wherein the water contained in the initial emulsion has left the product by sublimation.

The lyophilised makeup product, free from water, conserves substantially the same volume as it previously occupied, for example, in a compact, prior to its lyophilisation. The lyophilised makeup product of the present disclosure advantageously has a low density, less than 1 g/cm³, and preferably less than 0.5 g/cm³ and even more preferably less than 0.4 g/cm³.

In particular, the density of the lyophilised makeup product of the present disclosure is lower than the density of a product obtained by evaporation of the base composition. On example, by way of non-restrictive illustration, the lyophilised product obtained by lyophilisation of the base composition described in Example 1 has a density of around 0.34 g/cm³, whereas the product obtained by evaporation of the base composition described in Example 1 has a density of 0.54 g/cm³.

Furthermore, the lyophilisation permits the product to conserve an appearance and structure similar to those prior to the lyophilisation process, which is to say before the water leaves the product. On the contrary, the evaporation causes major modifications to the appearance, such as the loss of volume, retraction or even cracking of the product.

Traditional powders, obtained by the classic compacting technology, also have a higher density than that of the lyophilised makeup product of the present disclosure, and most often higher than 1 g/cm³.

The cavities formed in the lyophilised makeup product, thanks to the presence in sufficient quantities of hydrocolloids dissolved in the aqueous phase, advantageously form a network for re-hydrating the product. A lyophilised makeup product is thus obtained with high porosity in comparison with classic products obtained by the evaporation of emulsions or by the compacting of powders.

In one particularly interesting embodiment of the present disclosure, the lyophilised makeup product comprises a proportion of predominantly insoluble fillers of more than 60% in weight, and preferably of more than 65% in weight. Even more preferably, the lyophilised makeup product of the present disclosure comprises a proportion of mother of pearl particles of more than 40% in weight, and even more preferably 60% in weight. By way of example, the makeup product obtained by lyophilisation of the base composition illustrated in Example 1 (Table 2) comprises a percentage of mother of pearl of more than 60%, of around 64%, and a percentage of predominantly insoluble fillers of more than 70%.

Even though the product has a high proportion of fillers (and especially mother of pearl), the lyophilised makeup product of the present disclosure has good mechanical properties and, in particular, good mechanical resistance to impacts. By way of illustration, a drop test was carried out on various makeup products, which is to say lyophilised eye shadow obtained by lyophilisation of the base composition described in Example 1 and a classic eye shadow, formed by compacted powders. These makeup products are contained in metal trays with square cross sections, of which the sides measure 18 mm. The drop test consists of dropping the trays from a height of 30 centimetres and counting how many falls the trays are capable of resisting before the product breaks. The results are shown in Table 7 below.

TABLE 7

| | % of mother | Max. number of falls |
|---|---|---|
| Makeup product | of pearl | before breakage |
| Lyophilised eye shadow | 65 | >30 |
| of example 1 | | |
| “Compacted powders” | ≈20 | ≈7 |
| classic eye shadow | | |

It may be seen that even if the lyophilised makeup product of the present disclosure has a high percentage of mother of pearl, in comparison to classic products, the product has an impact resistance that is far greater than that of classic eye shadows.

Furthermore, as soon as the percentage of mother of pearl increases in the classic makeup products, the production and, in particular, the compacting operation of the powders becomes complicated or even impossible to carry out, given the quality and productivity requirements at industrial scale. In other terms, the traditional techniques do not permit makeup products to be obtained that are stable and resistant with a high percentage of mother of pearl, especially a percentage of mother of pearl of more than 60%. Furthermore, even for lower percentages of mother of pearl, the classic makeup products are less resistant to impacts than the lyophilised makeup product of the present disclosure.

This arises especially from the fact that the mother of pearl particles are lamellar particles which cannot be organised in to a compact network, during the compacting operation, contrary to other insoluble fillers, such as talc. Advantageously, the expanded structure of the lyophilised makeup product of the present disclosure forms a cement in which there are cavities inside which the fillers, and especially the mother of pearl particles, are imprisoned. The fillers, thus trapped and distributed regularly throughout the cavities, do not form clusters that are likely to create, locally, fragile zones and breakage points in the product.

Advantageously, the lyophilised makeup product of the present disclosure is formed by an eye shadow, blusher, foundation cream or lipstick.

Examples of formulations of the base composition permitting, for example, lyophilised makeup products of the present disclosure to be obtained, are described in more detail in the Examples 1-3 mentioned above.

The present disclosure also relates to a process for manufacturing a lyophilised makeup product including:

a step (a) of preparation of a base composition or of an intermediate composition obtained by dilution of the base composition, the composition comprising an emulsion in aqueous phase and a oily phase as well as predominantly insoluble fillers in suspension in the emulsion, wherein the composition has a proportion of fillers greater than or equal to 50% in weight;

a step (b) of lyophilisation of the base composition;

Step (a) includes a sub-step of incorporation of dispersing agents in the emulsion in sufficient quantity to form, during the step (b) of lyophilisation, a quality network capable of trapping the fillers.

Step (a) of preparation of a base composition advantageously comprises a first manufacturing step of a dry mixture composed of fillers, and, in particular, pigments and mother of pearl. This manufacturing step is comparable to that of a classic cosmetic powder.

Step (a) of preparation also comprises a second manufacturing step of an emulsion, containing the oily phase and the aqueous phase as well as dispersing agents, and, more precisely, hydrocolloids in the aqueous phase and possibly powders in suspension. This manufacturing step is substantially identical to that of a classic emulsion.

Step (a) of preparation then comprises a third step of mixing the emulsion with the dry mixture, so as to obtain a thick paste, which is in the form of aggregates and which forms the base composition previously described, which includes a proportion of fillers that is greater than or equal to 50% in weight. The base composition is then introduced, in pure form or diluted in water, into the trays, so that the base composition may be lyophilised.

Lyophilisation is a technique which permits a product to be dried by sublimating the water it contains. Step (b) of lyophilisation previously mentioned advantageously comprises a first sub-step in which the base composition or the intermediate composition is chilled to a temperature of less than −10° C., and preferably between −20 and −80° C., to freeze or deep freeze it, preferably for a period of at least one hour, and, for example, for a period of 2 to 10 hours.

Step (b) of lyophilisation then includes a second sub-step in which the frozen product thus obtained is placed in a vacuum, corresponding to an absolute pressure preferably greater than 10 Pa, and preferably less than 100 Pa, maintaining the product at a temperature of less than −5° C., and preferably between −15 and −40° C., to dehydrate the product, by sublimation, preferably for several hours, for example, from 10 to 50 hours. The ice, which forms the most volatile phase of the product, is thus transformed directly into steam without passing by the liquid phase.

Step (b) of lyophilisation then includes a third sub-step in which secondary dehydration is caused, which is to say the desorption of the product by progressive heating of the latter up to a positive temperature of less than 50° C., and preferably between 20 and 50° C., for several hours, for example, for between 5 and 30 hours.

In one particularly advantageous method, the proportion by weight of the concentration between the dispersing agents and the fillers ([dispersing agents]/[fillers]) is between 0.01 and 0.5, and preferably between 0.01 and 0.1, in order to obtain satisfactory dispersion of the fillers in the base composition, and thus a homogenous product, and also to form a network of cavities capable of trapping the fillers.

The manufacturing process of the present disclosure further advantageously comprises a step (c) of dilution, prior to the step (b) of lyophilisation, in which the base composition is diluted in a quantity of water that is sufficient so that the intermediate composition has a proportion of water that is greater than 50% in weight, and preferably that is greater than 60% in weight. This step (c) of dilution thus permits an intermediate composition to be obtained that is more fluid than the base composition and thus easier to spread onto the supports, and especially in the trays.

The dilution of the base composition also permits, after lyophilisation, a makeup product to be obtained that is much expanded, with a low density with respect to the classic makeup products, such as compacted powders.

The present disclosure also relates to a new use of dispersing agents in sufficient quantity inside a lyophilised makeup product obtained by lyophilisation of a composition comprising, on the one hand, an emulsion of a oily phase and an aqueous phase and, on the other hand, fillers that are at least partially insoluble, in suspension in the emulsion and representing 50% in weight of the composition, to obtain by lyophilisation a network of cavities capable of trapping the fillers.

Preferably, and as previously described, the dispersing agents are advantageously formed by hydrocolloids with a high dispersing power, preferably selected from polyacrylamide isoparaffin laureth-7 and/or magnesium aluminium silicate and/or xanthan gum.

The proportion by weight between the dispersing agents and the fillers is between 0.01 and 0.5, and preferably between 0.01 and 0.1, as described previously.

The use of dispersing agents thus permits a lyophilised makeup product to be obtained with a porous and expanded structure with cavities capable of trapping and imprisoning when dry predominantly insoluble fillers, present in a high percentage (greater than or equal to 50%).

The lyophilised makeup product of the present disclosure is therefore capable of containing a high percentage of insoluble fillers, and, in particular, mother of pearl, while conserving good mechanical resistance, especially to impacts, without its structure being made fragile by the presence of fillers.

Another advantage of the lyophilised makeup product of the present disclosure arises from the fact that the liberation of the fillers, and especially the pigments, takes place progressively, as the product is applied or re-hydrated, and homogeneously, thanks to the homogenous distribution of the fillers in the cavities.

Another advantage of the lyophilised makeup product of the present disclosure arises from the fact that its manufacturing steps may be successively carried out on different production sites, and especially on a first production site, where the base composition is prepared, then on a second production site, where, on the one hand, the intermediate composition is prepared, by dilution of the base composition, and, on the other hand, the lyophilisation of the intermediate composition. Thanks to the structure of the base composition, which is in the form of solid aggregates, the base composition is furthermore easy to package and transport from the first production site to the second production site. Once lyophilised, the makeup product of the present disclosure is preferably taken to another production site so that the makeup product may be packaged.

The invention described in the present disclosure can be used in the manufacture and use of compositions for cosmetic purposes.

All patents, patent applications and publications referred to herein are incorporated by reference in their entirety.

1. A base composition for makeup capable of being lyophilised to form a makeup product, the base composition comprising:
a) an emulsion of an oily phase and an aqueous phase;
b) fillers which are predominantly insoluble; and
c) dispersing agents, in sufficient quantity to ensure the dispersion of the fillers in the emulsion and to form, after lyophilisation, a network of cavities capable of trapping the fillers;
the base composition including a proportion of fillers greater than or equal to 50%.

2. The base composition of claim 1, wherein the dispersing agents are formed by hydrocolloids.

3. The base composition of claim 2, wherein the hydrocolloids are selected from the group consisting of polyacrylamide isoparaffin laureth-7, magnesium aluminium silicate, and xanthan gum.

4. The base composition of claim 1, wherein the proportion by weight between the dispersing agents and the fillers is between 0.01 and 0.5.

5. The base composition of claim 1, wherein the proportion by weight between the dispersing agents and the oily phase is between 0.01 and 1.

6. The base composition of claim 1, wherein the proportion by weight between the oily phase and the fillers is between 0.1 and 3.5.

7. The base composition of claim 1, wherein the fillers are selected from one or more of the following groups consisting of
a) mineral or organic, natural or synthetic pigments;
b) lacquers;
c) mother of pearl particles;
d) texturing effect fillers; and
e) optical effect fillers.

8. The base composition of claim 1, further comprising at least one active principle.

9. The base composition of claim 8, wherein the active principle is selected from one or more of the following groups consisting of
a) instant tanning lotions;
b) anti UVA and anti UVB sun screens and filters;
c) anti-radicals;
d) bleaching agents;
e) hydrating agents; and
f) treating agents.

10. The base composition of claim 1, wherein the emulsion is formed by an emulsion of oil in water or water in oil and/or by an emulsion of silicone in water or water in silicone.

11. The base composition of claim 1, further comprising a proportion of water of less than 50% by weight.

12. The base composition of claim 1, further comprising a proportion of oily phase of less than 10% by weight.

13. An intermediate composition for makeup capable of being lyophilised to form a makeup product and including a mixture comprising:
a) a base composition comprising
i) an emulsion of an oily phase and an aqueous phase,
ii) fillers which are predominantly insoluble, and
iii) dispersing agents, in sufficient quantity to ensure the dispersion of the fillers in the emulsion and to form, after lyophilisation, a network of cavities capable of trapping the fillers, the base composition including a proportion of fillers greater than or equal to 50%; and
b) a quantity of water sufficient so that the intermediate composition has a proportion of water greater than 50% by weight.

14. The intermediate composition of claim 13, wherein the intermediate composition has a viscosity of between 100 and 150 mPa·s at 25° C.

15. The intermediate composition of claim 13, wherein the intermediate composition is thermodynamically unstable.

16. A lyophilised makeup product obtained by lyophilisation of a base composition comprising: a) an emulsion of a oily phase and an aqueous phase; b) fillers which are predominantly insoluble; and c) dispersing agents, in sufficient quantity to ensure the dispersion of the fillers in the emulsion and to form, after lyophilisation, a network of cavities capable of trapping the fillers; the base composition comprising a proportion of fillers greater than or equal to 50%.

17. The product of claim 16, wherein the fillers comprise more than 60% by weight.

18. The product of claim 16, wherein the product has a density of less than 1 g/cm³.

19. The product of claim 16, wherein the product is formed either as an eye shadow, blusher, foundation cream or lipstick.

20. A process for manufacturing a lyophilised makeup product, comprising:
a) preparing either a base composition or an intermediate composition obtained by dilution of the base composition, the composition comprising an emulsion in aqueous phase and a oily phase as well as fillers which are predominantly insoluble in suspension in the emulsion, wherein the base composition has a proportion of fillers greater than or equal to 50% by weight; and
b) lyophilizing either the base composition or the intermediate compositions;
wherein step a) includes a sub-step of incorporating dispersing agents in the emulsion in sufficient quantity to form, during step b), a network capable of trapping the fillers.

21. The process of claim 20, wherein the dispersing agents are formed by hydrocolloids.

22. The process of claim 21, wherein the dispersing agents are selected from the group consisting of polyacrylamide isoparaffin laureth-7, magnesium aluminium silicate, and xanthan gum.

23. The process of claim 20, wherein the proportion by weight of the concentration between the dispersing agents and the fillers is between 0.01 and 0.5.

24. The process of claim 20, further comprising a step c) diluting, prior to the step b) the base composition in a quantity of water that is sufficient so that the intermediate composition has a proportion of water that is greater than 50% by weight.

25. A lyophilised makeup product obtained by lyophilisation of an intermediate composition comprising: a) a base composition comprising: i) an emulsion of a oily phase and an aqueous phase; ii) fillers which are predominantly insoluble; and iii) dispersing agents, in sufficient quantity to ensure the dispersion of the fillers in the emulsion and to form, after lyophilisation, a network of cavities capable of trapping the fillers; the composition including a proportion of fillers greater than or equal to 50%; and b) a quantity of water sufficient so that the intermediate composition has a proportion of water greater than 50% by weight.
